(19) United States
(12) Patent Application Publication
(10) Pub. No.: US 2010/0010375 A1
(43) Pub. Date: Jan. 14, 2010

(54) DEVICE AND METHOD FOR ANALYZING BODY FLUIDS

(76) Inventors: HANS-PETER HAAR, Wiesloch (DE); Hans List, Hesseneck-Keilbach (DE)

Correspondence Address: BOSE MCKINNEY & EVANS LLP, 111 Monument Circle, Suite 2700, INDIANAPOLIS, IN, 46204, US

(21) Appl. No.: 12/479,778

(22) Filed: Jun. 6, 2009

Related U.S. Application Data
(63) Continuation of application No. PCT/EP2007/063130, filed on Dec. 3, 2006.

(30) Foreign Application Priority Data
Dec. 7, 2006 (EP) EP 06025269.9

(51) Int. Cl. A61B 5/151; G01N 33/50; A61L 2/08

(52) U.S. Cl. 600/583; 73/61.41; 422/22

(57) ABSTRACT

A device for analyzing body fluids comprising a lancing element that can puncture a body part along a lancing axis. The lancing element has a collection volume for collecting body fluid obtained by a puncture and a receiving element that can be loaded with body fluid from the collection volume and that can detect a component of the body fluid. The lancing element and the receiving element are arranged in the lancing axis so that they can be moved relative to one another such that the receiving element dips into the collection volume in the lancing direction during a transfer phase following the puncture.

### RELATED APPLICATIONS

This application is a continuation application of International Application PCT/EP2007/063130, filed Dec. 3, 2007, which claims priority to EP 06025269.9, filed Dec. 7, 2006, which are hereby incorporated by reference in their entirety.

### BACKGROUND

The invention concerns a device for analyzing body fluids, especially for blood sugar tests, comprising a lancing element that can puncture a body part along a lancing axis. The lancing element has a collection volume for collecting body fluid obtained by a puncture, and a receiving element that can be loaded with body fluid from the collection volume. The receiving element can detect a component of the body fluid. The invention additionally concerns a corresponding analytical method and a method for sterilizing such a device designed as a disposable part.

A test element for examining body fluids for analytical purposes and especially for determining the blood glucose concentration is described in an earlier application WO 2008/068215 A2 of the applicant. This application teaches a lancing element with a collection area for body fluid where the collection area is formed by a collecting opening which is elongated in the direction of lancing and a permanently integrated light guide, the distal end of which is located in a proximal measuring zone of the collecting opening. This allows an optical detection of an analyte to be carried out by means of a microfluidic liquid transfer within a lancing structure.

### SUMMARY OF THE INVENTION

The present invention further develops the systems known in the prior art and optimizes a device and a method of the type stated above by providing reliable sample processing where the aim is for the smallest possible amounts of sample, reducing the lancing pain, and also simplifying production.

Embodiments incorporating the invention are based on the idea of avoiding an excess volume for a capillary sample transport and instead bringing the receiving element into direct contact with the sample by a suitable relative movement. Accordingly, the lancing element and the receiving element can be arranged in or along the lancing axis so that they can be moved relative to one another. The receiving element can be arranged in an initial position outside the collection volume of the lancing element such that the receiving element does not come into body contact during the puncture and such that the receiving element extends or dips into the collection volume in the lancing direction during a transfer phase following the puncture. As a result, the receiving element engages in the collection volume in a receiving position. This allows the receiving element to be adequately wetted with liquid even without capillary transport of the liquid, and malfunctions are avoided to a large extent by the directed immersion. Hence, it is possible to carry out a complete measurement process to a large extent automatically with very small amounts of samples in a miniaturized design. In this connection, the receiving element can either merely mediate a further sample transport or act directly as a sensory element.

In one embodiment, the distance between the lancing member of the lancing element and the receiving element is smaller in the transfer phase than in a prior lancing phase. Hence, body contact of the receiving element is avoided in the lancing phase while the amount of sample can be correspondingly reduced by the reduction in distance.

The receiving element can be advantageously inserted into the collection volume by a retracting movement of a lancing drive coupled with the lancing element. Alternatively, it is also possible that the receiving element can be moved into the collection volume by means of a feed drive.

In order to be able to utilize drive units that are permanently mounted in the device, it is advantageous when the lancing element and/or the receiving element have a docking structure for a detachable drive coupling at one proximal end.

It is advantageous for a simplified measurement process when the lancing element is mounted in a linear guide that can be formed by an associated case.

A constructionally advantageous embodiment provides that the receiving element engages in a recess of the lancing element leading to the collection volume.

The lancing element and the receiving element can be advantageously designed to belong together as a disposable part. This allows measurements to be carried out with great user convenience. A further improvement in this regard can be achieved by a magazine and in particular a disk, drum or stack magazine designed to receive a plurality of lancing elements and associated receiving elements.

In order to further increase the measurement integration it is advantageous when the receiving element has a measuring member for an optical or electrochemical measurement in the collected body fluid. In this connection it is preferred that the measuring member is coated with a test reagent that reacts with the component of the body fluid while under liquid contact.

For signal transmission to an instrument unit it is advantageous when the receiving element can be connected or is connected to a measuring unit via a light guide routed in the lancing element.

For an optimized uptake of liquid, it is advantageous when the collection volume is formed by a slot that is open on both sides or a channel of the lancing element that is open on one side.

Another aspect of the invention is that the collection volume may receive or require less than 50 nl, preferably less than 10 nl body fluid. In this manner it is possible to further reduce the pain during sample collection and thus increase user acceptance. Due to the special sample collection, it is sufficient when an aliquot of the body fluid is applied from the collection volume onto a distal front side of the receiving element where the aliquot can be less than 5 nl and preferably less than 1 nl.

The invention also concerns a lancing apparatus for use of at least one device in the form of a disposable part comprising a lancing drive that can be triggered by a user where, in the unused state, the disposable part is mounted in a first position inside the apparatus and after the puncture it is stored in a used state in a second position at a distance thereto inside the apparatus. Hence, this ensures that they are provided and disposed of without user manipulation.

In a process according to the invention the receiving element are moved in the lancing axis relative to one another during a transfer phase that follows the puncture such that the receiving element dips into the collection volume in the direction of lancing. This also results in the advantages that have already been elucidated above.

In an advantageous embodiment the distance between the receiving element and a distal lancing member of the lancing element is reduced in the transfer phase and the receiving element is moved together with the lancing element during the puncture or the lancing element is driven in a reciprocating lancing movement while the receiving element is held stationary.

The lancing element is advantageously located outside the body part containing the body fluid in the transfer phase and it is of further advantage when an optical or electrochemical measurement is carried out in the collected body fluid by means of the receiving element as a measuring member.

A further aspect of the invention is a method for sterilizing a device according to the invention designed as a disposable part in which the receiving element coated with a test reagent is kept at a distance from a distal lancing member of the lancing element and the lancing member is preferably irradiated with an electron beam. In this connection, it is possible that the radiation and in particular the electron beam is focused on the lancing member and/or the distance between the receiving element and the lancing member is selected such that the test reagent is not damaged by the radiation.

### BRIEF DESCRIPTION OF THE DRAWINGS

The above-mentioned aspects of the present invention and the manner of obtaining them will become more apparent and the invention itself will be better understood by reference to the following description of the embodiments of the invention, taken in conjunction with the accompanying drawings, wherein:

FIG. 1a is a diagrammatic view of a device for blood sugar measurement with a disposable lancing and detection element;

FIGS. 1b and c show the lancing and detection element of FIG. 1a in a lancing and detection position;

FIGS. 2a-2c show a disk magazine with an active lancing and detection element in various positions in a top-view;

FIGS. 3a-3b show a further embodiment of a lancing and detection element in two positions in a perspective views; and

FIGS. 4a-4e show yet a further embodiment of a lancing and detection element in various positions in axial section views.

### DETAILED DESCRIPTION

The embodiments of the present invention described below are not intended to be exhaustive or to limit the invention to the precise forms disclosed in the following detailed description. Rather, the embodiments are chosen and described so that others skilled in the art may appreciate and understand the principles and practices of the present invention.

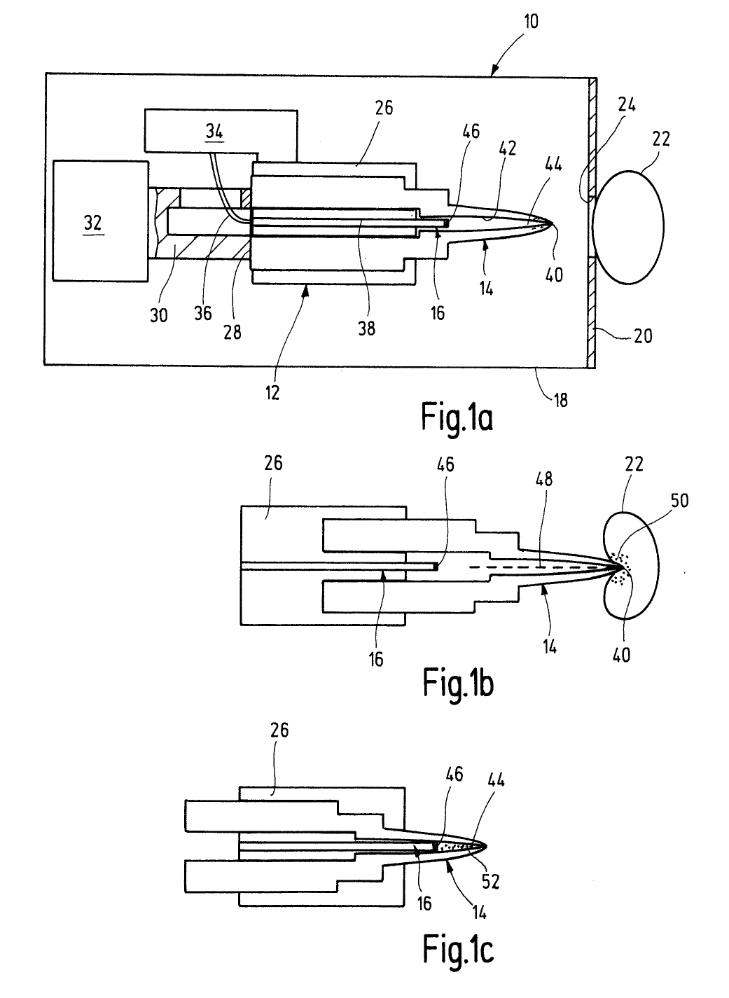

The diagnostic measuring arrangement shown in FIG. 1 comprises an analyzer 10 for blood sugar measurements and at least one test element 12 that can be inserted therein as a consumable for single use and which has a lancing element 14 and a receiving or detection element 16.

As shown in FIG. 1a, the analyzer 10 has a device housing 18 with a support 20, for example, for a finger 22 (a portion of which is shown) in the area of a piercing opening 24. The lancing element 14 is mounted within the housing 18 in a linear guide 26 in order to execute a reciprocating lancing movement against the applied finger 22. For this purpose the lancing element 14 can be detachably connected to the pusher 30 of a lancing drive 32 via a proximal docking structure 28. A detection unit 34 which can be coupled via a flexible lead 36 to a light guide 38 of the detection element 16 is provided inside the device for an optical glucose detection in a blood sample obtained by means of the lancing element 14.

The lancing element 14 is formed as a flat part, for example, from a stainless steel sheet and has a distal tip 40 as a lancing member in the rear area of which there is a collection volume 44 delimited by a slot 42 that is open on both sides.

The light guide of the detection element 16 engages in the slot 42, and a front face or receiving element of the light guide 38 coated with a test reagent 46 is aligned head-on to the collection volume 44. The test reagent 46 changes color when blood fluid is applied to it due to the glucose that is contained therein so that it is possible on the basis of the color change to carry out an optical detection by means of the detection unit 34 via the light guides 38, 36. The measuring result can be displayed to the user in order to enable an on the spot self-examination.

As shown in FIGS. 1a, b, and c, the lancing element 14 is arranged in the guide 26 such that it can be substantially moved linearly relative to the detection element 16 that is held in a fixed position in the device. Hence, the lancing element 14 can be moved backwards and forwards along a lancing axis 48 so that the body part 22 can be punctured and the blood sample obtained in this process can be transferred onto the test reagent 46. In the initial position of FIG. la the lancing movement can be triggered by the user after applying the finger 22. In the subsequent lancing phase, the lancing member 40 penetrates into a blood-yielding skin layer 50 when the lancing depth has been suitably adjusted. During the retraction movement which generally takes place more slowly than the rapid forwards movement during the puncture, body fluid 52 (blood or also tissue fluid) is taken up in the collection volume 44. A microscopic amount of blood in the range of 10 nanoliters or less is sufficient in this case. In the final transfer phase, a portion of the collected body fluid 52 is transferred onto the detection element 16 by the return movement of the lancing element 14 and the reagent layer 46 as a sensory element dips proximally into the collection volume 44. Due to the reduction in the distance between the sensor element (test reagent 46) and the lancing member 40 or the collection volume 44, it is thus not necessary to take up any excess liquid volume for a capillary transport of the body fluid. An extremely small partial volume of about 1 nanoliter is sufficient for the actual detection reaction at the tip of the light guide.

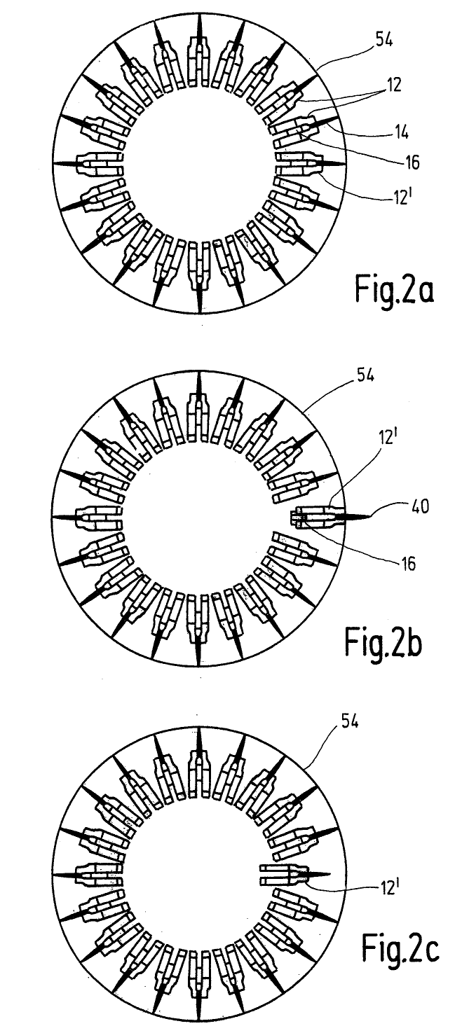

FIGS. 2a-2c illustrate a possible use of a large number of test elements 12 in a disk magazine 54. The test elements can be sequentially brought into an active position (test element 12′) by rotating such a disk magazine 54 in a device 10. According to FIG. 2b the puncture then takes place while increasing the distance between the lancing member 40 and the detection element 16 whereas in the transfer phase according to FIG. 2c liquid is transferred by reducing the distance. The used test element is subsequently disposed of by advancing the magazine 54.

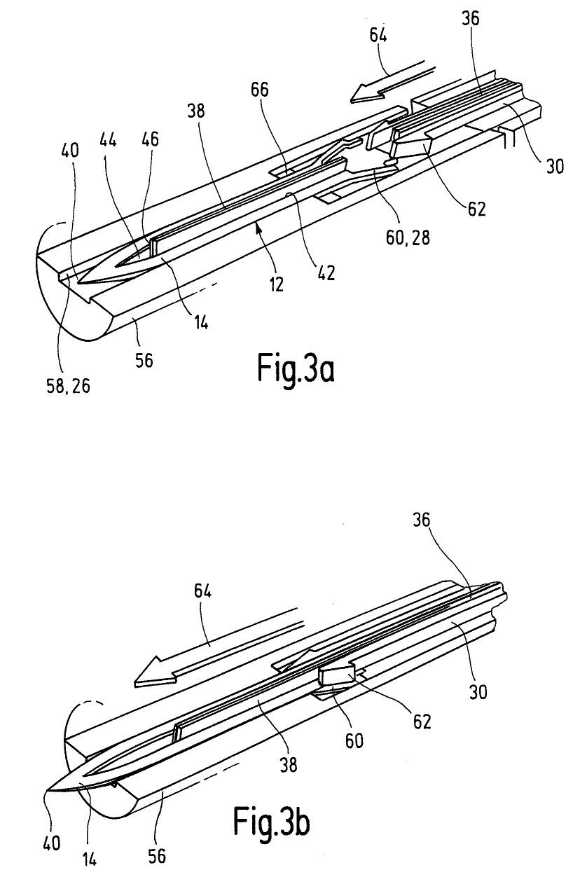

FIGS. 3a and 3b show an embodiment of a test element 12 in an accompanying cylindrical case 56 which forms a part of the disposable article and together with a central recess 58 forms a guide 26 for the U-shaped longitudinally slotted lancing element 14. A light conducting rod 38 arranged in the slot 42 of the lancing element 14 is also permanently connected to the case 56 as a part of the consumable article. The front wall of the light conducting rod 38 is coated with the test reagent 46 and the rear side can be docked to the light guide 36 which is coupled on the instrument side to the detection unit 34. For this purpose a gripper-like claw clutch 60 is provided on the lancing element 14 as a docking structure 28 which automatically engages in a form-fitting manner with the head piece 62 of the drive pusher 30 during the advance movement (arrow 64). For this purpose run-on slopes 66 are formed on the inside of the case 60 which close the elastically spreading claws 60 so that it is possible to execute a return movement after blood collection. In this embodiment it is possible that the case 56 is moved up to the finger 22 by a separate advancing drive and in doing so the free front end presses against the skin as a reference surface for the lancing depth and stabilizes the skin.

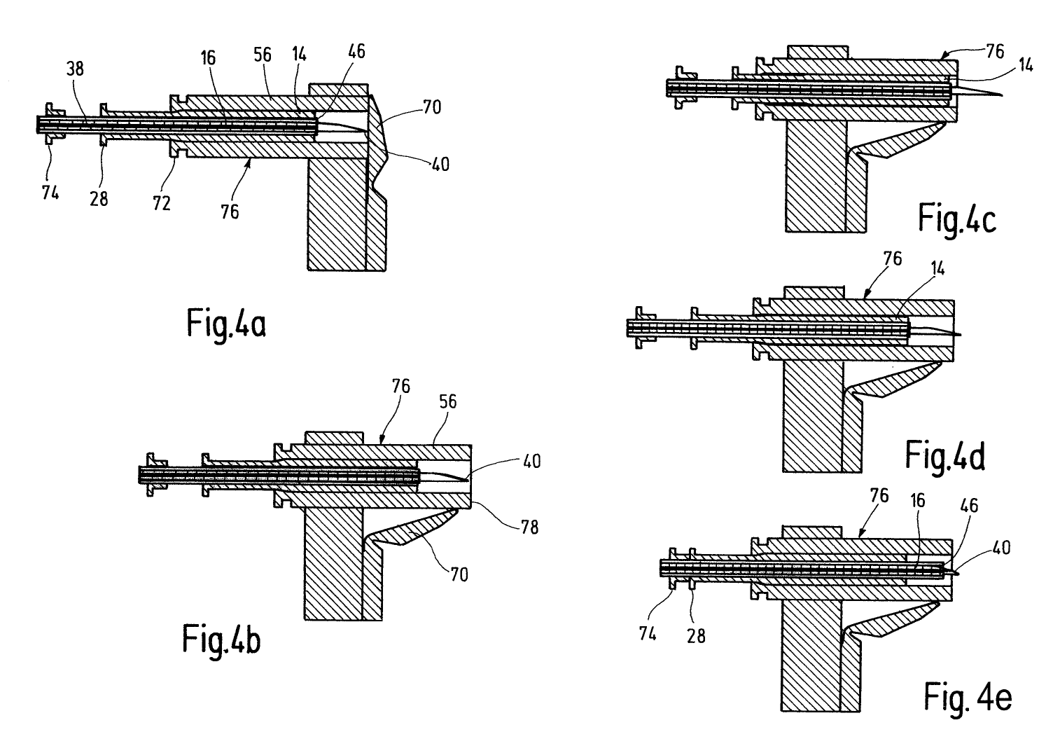

In the embodiment shown in FIGS. 4a-4e, the same parts are also provided with the same reference numerals as described above. In the initial position of FIG. 4a the front opening of the case 56 is closed in a sterile manner by a sealing cover 70. The test reagent 46 thus remains also protected against environmental influences and in particular against moisture. The case 56, the lancing element 14 and the detection element 16 are each provided at their proximal end with a docking structure 72, 28, 74 for a separate drive coupling. In the sequence of operations that are shown, the sealing cover 70 is first opened by advancing the entire disposable part 76 and the front end makes contact with the skin (FIG. 4b). When the cover 70 is pushed open by the front end 78 of the case 56, the sensitive lancing tip 40 is protected from damage. This is followed by the lancing advance of the lancing element 14 up to the maximum penetration depth (FIG. 4c) and the subsequent return movement (FIG. 4d). A separate advance movement of the detection element 16 then takes place in the lancing axis defined thereby such that the test reagent 46 at the front end comes nearer to the lancing member 40 and dips into the collection volume 44 (FIG. 4e).

In the puncture according to FIG. 4c the relatively large distance between the front face or receiving element of detection element 16 and lancing member 40 protects the test person from contamination with the test reagent 46. Furthermore, this distance also allows a simplified sterilization of the lancing tip 40 during manufacture. In this case an electron beam can be focussed onto the tip 40 without the radiation damaging the test reagent 46 located at a suitable distance or making it unusable. In particular special barriers between the test reagent and lancing tip can be dispensed with.

While exemplary embodiments incorporating the principles of the present invention have been disclosed hereinabove, the present invention is not limited to the disclosed embodiments. Instead, this application is intended to cover any variations, uses, or adaptations of the invention using its general principles. Further, this application is intended to cover such departures from the present disclosure as come within known or customary practice in the art to which this invention pertains and which fall within the limits of the appended claims.

1. A device for analyzing body fluids which is especially useful for blood sugar tests, comprising a lancing element that moves during a lancing phase along a lancing axis to puncture a body part, the lancing element having a collection volume for collecting body fluid obtained by a puncture and a receiving element that can be loaded with body fluid from the collection volume, the receiving element detecting a component of the body fluid during use of the device, wherein the lancing element and the receiving element are moveable relative to one another along the lancing axis and the receiving element is arranged in an initial position away from the collection volume such that the receiving element does not contact the body part during the puncture, and wherein the receiving element enters the collection volume during a transfer phase following the lancing phase.

2. The device of claim 1, wherein the lancing element has a distal lancing member, the distance between the lancing member and the receiving element being smaller in the transfer phase than in the lancing phase.

3. The device of claim 1, further comprising a lancing drive coupled to the lancing element, the receiving element being movable into the collection volume by a retracting movement of the lancing drive.

4. The device of claim 1, further comprising a feed drive which moves the receiving element into the collection volume.

5. The device of claim 1, wherein the lancing element or the receiving element have a docking structure for a detachable drive.

6. The device of claim 1, further comprising a linear guide in which the lancing element is mounted.

7. The device of claim 1, wherein the lancing element comprises a recess leading to the collection volume, the receiving element engaging the recess.

8. The device of claim 1, wherein the lancing element and the receiving element together comprise a disposable part.

9. The device of claim 8, further comprising a magazine having a plurality of lancing elements and associated receiving elements.

10. The device of claim 9, wherein the receiving element comprises a measuring member for an optical or electrochemical measurement in the collected body fluid.

11. The device of claim 10, wherein the measuring member is coated with a test reagent that reacts with the component of the body fluid while under liquid contact.

12. The device of claim 10, wherein the receiving element is connectable to a measuring unit via a light guide routed in the lancing element.

13. The device of claim 1, wherein the collection volume comprises a slot or channel of the lancing element.

14. The device of claim 1, wherein the collection volume has a capacity of less than 10 nl body fluid.

15. The device of claim 1, wherein, during use of the device, an aliquot of the body fluid is transferred from the collection volume onto a distal front side of the receiving element, the aliquot being less than 5 nl.

16. The device of claim 1, further comprising a lancing drive that can be triggered by a user.

17. The device of claim 1, further comprising a measuring unit that interacts with the receiving element to detect the component of the body fluid.

18. A method for analyzing body fluids in a device of the type having a lancing element comprising a collection volume and a receiving element, the method comprising:
moving the lancing element along a lancing axis and into a body part during a lancing phase;
drawing body fluid into the collection volume;
moving the receiving element and the collection volume relative to one another along the lancing axis during a transfer phase to bring the receiving element in contact with body fluid in the collection volume;
loading the receiving element with a portion of the body fluid; and
testing the body fluid loaded on the receiving element for the presence or concentration of a component of interest.

19. The method of claim 18, wherein the distance between the receiving element and a distal lancing member of the lancing element is reduced in the transfer phase.

20. The method of claim 18, wherein the receiving element is moved together with a lancing member of the lancing element during the lancing phase.

21. The method of claim 18, further comprising driving a lancing member of the lancing element in a reciprocating lancing movement while holding the receiving element stationary.

22. The method of claim 18, wherein the lancing element is located outside the body part containing the body fluid in the transfer phase.

23. The method of claim 18, wherein the testing comprises an optical or electrochemical measurement.

24. A method of sterilizing a disposable lancing element of the type comprising a lancing member defining a collection volume therein and a receiving element coated with a reagent that produces a detectible response when contacted with a biological fluid having a component of interest, the receiving element and the collection volume being movable relative to one another along a lancing axis of the lancing member, the method comprising maintaining the receiving element at a distance from the lancing member of the lancing element and irradiating the lancing member.

25. The method of claim 24, further comprising selecting the distance to be sufficiently large such that the test reagent is not damaged by the irradiation.

26. The method of claim 24, wherein the radiation comprises an electron beam, the method further comprising focusing the electron beam on the lancing member such that the test reagent is not damaged by the radiation.

27. An apparatus for analyzing body fluids, comprising:
a housing;
a lancing element movable relative to the housing along a lancing axis to puncture a body part, the lancing element defining a collection volume in which body fluid is collected; and
a receiving element which produces a detectible response when contacted with body fluid having a component of interest, the receiving element and collection volume being movable relative to one another along the lancing axis between a first position in which the receiving element is remote from the collection volume and a second position in which the receiving element extends into the collection volume.

28. The apparatus of claim 27, further comprising a lancing phase during which the lancing element is moved along the lancing axis in a puncturing direction to puncture a body part followed by a transfer phase during which the collection volume and receiving element are moved from the first position to the second position.

29. The apparatus of claim 28, further comprising a lancing drive coupled to the lancing element, wherein the receiving element and collection volume are moved into the second position by a retracting movement of the lancing drive during the transfer phase.

30. The apparatus of claim 28, wherein the receiving element remains stationary relative to the housing during the puncture phase.

31. The apparatus of claim 28, wherein the receiving element moves with the lancing element during the puncture phase.

32. The apparatus of claim 27, further comprising a feed drive which moves the receiving element into the collection volume.

33. The apparatus of claim 27, further comprising a linear guide in which the lancing element is mounted.

34. The apparatus of claim 27, wherein the lancing element comprises a recess in which the receiving element is located, the receiving element being movable relative to the recess.

35. The apparatus of claim 27, wherein the receiving element comprises a measuring member for an optical or electrochemical measurement of collected body fluid.

36. The apparatus of claim 27, wherein the measuring member is coated with a test reagent that reacts with the component of the body fluid.

37. The apparatus of claim 27, further comprising a light guide connected to the receiving element.

38. The apparatus of claim 37, wherein the light guide is routed in the lancing element.

39. The apparatus of claim 27, wherein the collection volume comprises a slot or channel of the lancing element.
